(19) United States
(12) Patent Application Publication
(10) Pub. No.: US 2021/0143478 A1
(43) Pub. Date: May 13, 2021

(54) Electrolyte for Lithium Ion Batteries

(71) Applicant: Westfälische Wilhelms-Universität Münster, Münster (DE)

(72) Inventors: Stephan ROESER, Münster (DE); Johannes KASNATSCHEEW, Dülmen (DE); Ralf WAGNER, Münster (DE); Jaschar ATIK, Münster (DE); Gunther BRUNKLAUS, Münster (DE); Martin WINTER, Münster (DE)

(21) Appl. No.: 16/622,677

(22) PCT Filed: Jun. 13, 2018
(86) PCT No.: PCT/EP2018/065628

§ 371 Date: Dec. 13, 2019

(30) Foreign Application Priority Data
Jun. 14, 2017 (DE) 10 2017 113 141.8

(51) Int. Cl. H01M 10/0569; H01M 10/0525; C07C 43/13

(52) U.S. Cl. CPC H01M 10/0569; H01M 2300/0028; C07C 43/135; H01M 10/0525

(57) ABSTRACT

The disclosure relates to an electrolyte for an energy store comprising a conducting salt and a solvent. The solvent comprises at least one compound according to the general formula (1), as indicated in the following: wherein R¹, R², R³, R⁴ are, identically or independently of each other, selected from the group comprising linear or branched C₁₋₆-alkyl, C₂₋₆-alkenyl C₃₋₆-cycloalkyl and/or phenyl, each unsubstituted or mono- or polysubstituted by a substituent selected from the group comprising F, CN and/or C₁₋₂-alkyl, mono- or polysubstituted with fluorine.

### INTRODUCTION

The disclosure relates to the field of lithium ion batteries.

Lithium ion batteries (secondary batteries) are at present the leading technology in the field of rechargeable batteries, especially in the field of portable electronics. Conventional lithium ion batteries usually employ a graphite anode. Charge transport occurs via an electrolyte which comprises a lithium salt dissolved in a solvent. Various electrolytes and electrolyte salts are known in the prior art. Conventional lithium ion batteries at present usually employ lithium hexafluorophosphate (LiPF₆).

During operation of graphite anodes, reductive decomposition of the electrolyte occurs. The reaction products can form an adhering and electronically insulating but lithium ion-conducting film on the electrode. Suitable electrolytes induce the formation of a solid electrolyte interphase (SEI) on the electrode. The solid electrolyte interphase subsequently prevents the graphite from reacting further with the electrolyte and in this way protects the electrolyte against further reductive decomposition and the anode against destruction by cointercalation of the solvent. Especially when graphite anodes are used, the formation of such a film is necessary for reliable operation of the lithium ion battery. Without formation of a solid electrolyte interphase, the graphite anode is in the case of propylene carbonate-based electrolytes destroyed by the cointercalation of the solvent.

However, the reductive decomposition of the solvent propylene carbonate (IUPAC name 4-methyl-1,3-dioxolan-2-one) does not lead to formation of an effective solid electrolyte interphase. Instead, reductively induced gas evolution within the graphite layers, which is induced by cointercalation of propylene carbonate, brings about exfoliation and irreversible destruction of the active material. This limits the utilization of propylene carbonate despite its better thermal and physicochemical properties compared to ethylene carbonate (IUPAC 1,3-dioxolan-2-one) for lithium ion technology. Propylene carbonate can serve as model system for electrolytes which likewise display reductive decomposition without SEI formation and exfoliation of graphite.

Suppressing the exfoliation of graphite and the reductive decomposition of the solvent by use of highly concentrated electrolytes has already been proposed. However, the use of highly concentrated electrolytes, also known as “solvent-in-salt” electrolytes, is not economical since this approach requires a multiple of the normally required amount of electrolyte salt. In addition, the concentration (usually >3 mol l⁻¹) greatly increases the viscosity of the electrolyte, which leads to a marked decrease in the conductivity and the performance of the battery. Furthermore, it is to be expected that a decrease in the operating temperature results in the solubility product of the electrolyte salt going below the concentration of the electrolyte salt in the electrolyte solution, which leads to precipitation of the salt in the interior of the batteries. In addition, the density and therefore the total mass of the electrolyte increases at a constant volume and increasing addition of electrolyte salt. This likewise leads to the specific energy density (Ah kg⁻¹) of the battery as overall system decreasing.

Furthermore, the use of appropriate performance additives has been proposed. In commercial batteries in particular, vinylene carbonate (VC) and fluoroethylene carbonate (FEC) are relevant here. There is therefore a need for further agents which can prevent exfoliation of the graphite.

### SUMMARY

One object of the present disclosure, according to an embodiment, is to provide an electrolyte which overcomes at least one of the abovementioned disadvantages of the prior art. In particular, it is an object of the present disclosure, according to an embodiment, to provide a compound which assists the formation of a solid electrolyte interphase on graphite and thus makes reversible cycling of propylene carbonate-containing electrolytes possible.

This object is achieved, per an embodiment, by an electrolyte for an energy store, comprising an electrolyte salt and a solvent, wherein the solvent comprises at least one compound of the general formula (1) as indicated below:

[chemical structure]

where:
R¹, R², R³, R⁴ are identical or different and selected independently from the group comprising linear or branched C₁₋₆-alkyl, C₂₋₆-alkenyl, C₂₋₆-alkynyl, C₃₋₆-cycloalkyl and phenyl, in each case unsubstituted or singly or multiply substituted by a substituent selected from the group comprising F, CN and C₁₋₂-alkyl singly or multiply substituted with fluorine.

It has unexpectedly been found that tetraalkoxyethanes of the general formula (1) form a solid electrolyte interphase (SEI) on a graphite electrode. The use of tetraalkoxyethanes of the general formula (1) in electrolytes thus allows the use of graphite electrodes in solvents such as propylene carbonate which do not form an effective SEI on graphite. The tetraalkoxyethanes can here be used as sole solvent or as SEI additive or cosolvent for propylene carbonate-based electrolytes. Tetraalkoxyethanes of the general formula (1) can form a stable solid electrolyte interphase which can protect graphite anodes against exfoliation and a propylene carbonate electrolyte against continuous reductive decomposition over 300 charging and discharging cycles.

The term “C₁₋₆-alkyl” or “C₁-C₆-alkyl” encompasses, unless indicated otherwise, straight-chain or branched alkyl groups having from 1 to 6 carbon atoms. The term “C₃₋₆-cycloalkyl” refers to cyclic alkyl groups having from 3 to 6 carbon atoms. The terms “C₂₋₆-alkenyl” and “C₂₋₆-alkynyl” encompass, unless indicated otherwise, straight-chain or branched alkenyl or alkynyl groups having from 2 to 6 carbon atoms and in each case at least one double or triple bond.

The radicals R¹, R², R³ and R⁴ can be identical or different. The radicals R¹, R², R³ and R⁴ are preferably identical.

Preference is given to C₁-C₅-alkyl groups. Preferred C₁-C₅-alkyl groups encompass, unless indicated otherwise, straight-chain or branched alkyl groups having from 1 to 5 carbon atoms, preferably selected from the group comprising methyl, ethyl, propyl, isopropyl, butyl, isobutyl, pentyl, isopentyl and neopentyl.

The alkyl, alkenyl or alkynyl groups can be unsubstituted or singly or multiply, for example doubly or triply, substituted. Here, the alkyl, alkenyl or alkynyl groups can be multiply substituted on various, preferably on identical, carbon atoms. The substituent can be fluorine or CN (nitrile). In embodiments in which the groups R¹, R², R³, R⁴ are substituted, these are preferably substituted by fluorine, for example monofluorinated or multiply fluorinated or perfluorinated. C₃-C₆-Alkyl substituents in particular can bear a CF₃ group. Alkyl, alkenyl, alkynyl or cycloalkyl groups or phenyl can also be singly or multiply substituted by small fluorine-substituted C₁₋₂-alkyl groups, in particular by CF₃.

In certain embodiments, R¹, R², R³, R⁴ are identical or different and selected independently from the group comprising unsubstituted C₁-C₅-alkyl, preferably C₁-C₃-alkyl, or phenyl or C₁-C₅-alkyl, preferably C₁-C₃-alkyl, or phenyl singly or multiply substituted by fluorine, CN or CF₃.

Unsubstituted compounds, on the other hand, are usually cheaper and thus more economical as solvent or cosolvent in a lithium ion battery. Relatively small C₁-C₃-alkyl substituents in particular can be unsubstituted. In certain embodiments, R¹, R², R³, R⁴ are identical or different and selected independently from the group comprising methyl, ethyl, n-propyl and isopropyl, in particular from methyl and ethyl.

In certain embodiments, the compound of the general formula (1) is selected from among 1,1,2,2-tetramethoxyethane and 1,1,2,2-tetraethoxyethane. 1,1,2,2-Tetramethoxyethane is according to IUPAC nomenclature also referred to as tetramethyl 1,1,2,2-ethanetetracarboxylate, and 1,1,2,2-tetraethoxyethane is referred to as tetraethyl 1,1,2,2-ethanetetracarboxylate. 1,1,2,2-Tetramethoxyethane and 1,1,2,2-tetraethoxyethane have the formulae (2) and (3) below:

[chemical structure]

1,1,2,2-Tetramethoxyethane and 1,1,2,2-tetraethoxyethane in particular have been found to be very suitable as cosolvent for propylene carbonate for forming an effective SEI on graphite, which SEI effectively suppresses the cointercalation of propylene carbonate in graphite. 1,1,2,2-Tetramethoxyethane and 1,1,2,2-tetraethoxyethane in particular are therefore suitable as cosolvent or SEI additive or as sole solvent for lithium ion technology.

In some embodiments, the solvent can contain the compound of the general formula (1) in an amount of from ≥0.1% by weight to ≤100% by weight, based on the total weight of the electrolyte solvent. The tetraalkoxyethanes can be used as sole solvent. Furthermore, the tetraalkoxyethanes can be used as SEI additive. For example, the solvent can comprise the compound of the general formula (1) in an amount of from ≥0.1% by weight to ≤10% by weight, or from ≥1% by weight to ≤5% by weight, based on the total weight of the electrolyte solvent. The tetraalkoxyethanes can be used as cosolvent for propylene carbonate-based electrolytes. The electrolyte comprises the compound of the general formula (1) in an amount of from ≥10% by weight to ≤80% by weight, preferably in an amount of from ≥20% by weight to ≤50% by weight, particularly preferably in an amount of from ≥30% by weight to ≤50% by weight, based on the total weight of the electrolyte solvent. In an advantageous way, per an embodiment, proportions of, in particular, 30% by weight of 1,1,2,2-tetramethoxyethane or 1,1,2,2-tetraethoxyethane as cosolvent can effectively suppress the cointercalation of propylene carbonate in graphite. Here, the possibility of using comparatively small amounts of tetraalkoxyethane such as 1,1,2,2-tetramethoxyethane or 1,1,2,2-tetraethoxyethane makes this approach economical.

The electrolyte comprises at least one electrolyte salt, preferably a lithium salt, and a solvent comprising the compound of the general formula (1). Here, the compound of the general formula (1) can be the solvent. The electrolyte can also comprise a further solvent. In these embodiments, the compound of the general formula (1) functions as cosolvent. In other embodiments, the compound of the general formula (1) can be present in only small proportions and would then, in contrast to the further solvent present, be referred to as additive. The solvent serves as dissolution medium for the electrolyte salt or lithium salt. The terms solvent and dissolution medium will be used synonymously in the present text.

The electrolyte can contain a solvent selected from the group comprising unfluorinated or partially fluorinated organic solvents, ionic liquids, a polymer matrix and mixtures thereof. The electrolyte preferably comprises, in an embodiment, an organic solvent, in particular a cyclic or linear carbonate. In certain embodiments, the organic solvent is selected from the group comprising ethylene carbonate, ethyl methyl carbonate, propylene carbonate, dimethyl carbonate, diethyl carbonate, acetonitrile, propionitrile, 3-methoxypropionitrile, glutaronitrile, adiponitrile, pimelonitrile, gamma-butyrolactone, gamma-valerolactone, dimethoxyethane, 1,3-dioxolane, methyl acetate, ethyl acetate, ethyl methanesulfonate, dimethyl methylphosphonate, linear or cyclic sulfone, symmetrical or unsymmetrical alkyl phosphates and mixtures thereof.

In certain embodiments, the solvent is selected from the group comprising propylene carbonate, ethylene carbonate, ethyl methyl carbonate, dimethyl carbonate, diethyl carbonate and mixtures thereof. The electrolyte can, in particular, comprise solvents such as propylene carbonate which do not lead to formation of an effective solid electrolyte interphase. Preference is given, in an embodiment, to propylene carbonate and mixtures of propylene carbonate with ethylene carbonate, ethyl methyl carbonate, dimethyl carbonate and/or diethyl carbonate, in particular mixtures of propylene carbonate with dimethyl carbonate. When these solvents are used, an addition of the compounds according to the disclosure to form an effective solid electrolyte interphase may be particularly advantageous. 1,1,2,2-Tetramethoxyethane and 1,1,2,2-tetraethoxyethane may be particularly preferred as cosolvents.

For example, mixtures containing 50% by weight of 1,1,2,2-tetramethoxyethane and/or 1,1,2,2-tetraethoxyethane and 50% by weight of propylene carbonate, based on the total weight of the electrolyte solvent, may be preferred. Mixtures containing 1,1,2,2-tetramethoxyethane and/or 1,1,2,2-tetraethoxyethane and also propylene carbonate and dimethyl carbonate in a weight ratio of 1:1:1 or 1:2:2 may be likewise preferred. Such mixtures can have a good conductivity and bring about passivation of graphite electrodes.

A further advantage, according to an embodiment, in addition to the formation of an effective SEI is that the use of tetraalkoxyethanes contributes to an increase in the intrinsic safety of the electrolyte system by increasing the spontaneous ignition temperature compared to linear carbonates such as dimethyl carbonate and diethyl carbonate. Thus, 1,1,2,2-tetramethoxyethane and 1,1,2,2-tetraethoxyethane have a spontaneous ignition temperature of 47-53° C. and 71° C., respectively, while dimethyl carbonate and diethyl carbonate can ignite spontaneously at temperatures of 18° C. and 31° C., respectively. Furthermore, the temperature window in which the electrolyte is able to be used can be widened by use of 1,1,2,2-tetramethoxy ethane or 1,1,2,2-tetraethoxyethane as cosolvent. Thus, 1,1,2,2-tetramethoxyethane and 1,1,2,2-tetraethoxyethane have a melting point of −24° C. and −35° C., respectively, and a boiling point of about 155° C. and 196° C., respectively, while dimethyl carbonate and diethyl carbonate melt only at temperatures of 5° C. and −74° C., respectively, but boil at 91° C. and 126° C., respectively. Ethylene carbonate has a melting point of 36° C.

The electrolyte can also be a polymer electrolyte, for example selected from the group comprising polyethylene oxide, polyacrylonitrile, polyvinyl chloride, polyvinylidene fluoride, poly(vinylidene fluoride-co-hexafluoropropylene) and polymethyl methacrylate with addition of an electrolyte salt, or a gel polymer electrolyte comprising a polymer, an abovementioned organic solvent and/or an ionic liquid and an electrolyte salt. The electrolyte can likewise be formed by an ionic liquid and an electrolyte salt.

The electrolyte of the disclosure comprises at least one electrolyte salt, in particular a lithium salt, in addition to a solvent and at least one compound of the general formula (1). The electrolyte salt is preferably selected from the group comprising LiPF₆, LiBF₄, LiAsF₆, LiSbF₆, LiClO₄, LiPtCl₆, LiN(SO₂F)₂, LiN(SO₂CF₃)₂, LiN(SO₂C₂F₅)₂, LiC(SO₂CF₃)₃, LiB(C₂O₄)₂, LiBF₂(C₂O₄) and LiSO₃CF₃. The lithium salt is preferably selected from among LiN(SO₂CF₃)₂ (LiTFSI, lithium bis(trifluoromethanesulfonyl)imide, LiN(SO₂F)₂ (LiFSI) and LiPF₆. The concentration of the lithium salt in the electrolyte can be in conventional ranges, for example in the range from ≥1.0 M to ≤1.5 M. The use of relatively small amounts of electrolyte salt makes the electrolyte of the disclosure economical, in particular compared to “solvent-in-salt” electrolytes.

In an embodiment, the electrolyte comprises a compound of the general formula (1), in particular 1,1,2,2-tetramethoxyethane and/or 1,1,2,2-tetraethoxyethane, at least one lithium salt and propylene carbonate or a mixture of organic solvents comprising propylene carbonate. The electrolyte can, for example, be produced by mixing the compound of the general formula (1) with propylene carbonate or a solvent mixture containing propylene carbonate and introducing the lithium salt into the solvent.

The electrolyte can additionally contain at least one additive, in particular selected from the group comprising SEI formers, flame retardants and overcharging additives. For example, the electrolyte can contain a compound of the general formula (1) and also a further SEI former, for example selected from the group comprising fluoroethylene carbonate, chloroethylene carbonate, vinylene carbonate, vinylethylene carbonate, ethylene sulfite, propane sultone, propene sultone, sulfites, preferably dimethyl sulfite and propylene sulfite, ethylene sulfate, propylene sulfate, methylene methanedisulfonate, trimethylene sulfate, butyrolactones optionally substituted by F, C₁ or Br, phenylethylene carbonate, vinyl acetate and trifluoropropylene carbonate. For example, the electrolyte can contain a compound of the general formula (1) and also a further SEI former selected from the group comprising vinyl carbonate, fluoroethylene carbonate and ethylene sulfate. These compounds can improve the battery performance, for example the capacity, the long-term stability or the cycling life.

The compounds of the general formula (1), in particular 1,1,2,2-tetramethoxyethane and 1,1,2,2-tetraethoxyethane, are commercially available or can be prepared by methods with which a person skilled in the art will be familiar.

The electrolyte is suitable for a battery or a rechargeable battery, according to an embodiment, in particular as electrolyte for a lithium ion battery or a rechargeable lithium ion battery.

The present disclosure further provides an energy store, in particular electrochemical energy store, selected from the group comprising lithium battery, lithium ion battery, rechargeable lithium ion battery, lithium polymer battery, lithium ion capacitor and supercapacitor, comprising an electrolyte according to the disclosure.

For a description of the electrolyte, reference is made to the above description. The term “energy store” encompasses, for the purposes of the present disclosure, primary and secondary electrochemical energy storage devices, i.e. batteries (primary stores) and rechargeable batteries (secondary stores). In general language usage, rechargeable batteries are frequently referred to by the term “battery” which is frequently used as collective term. Thus, the term lithium ion battery is for the present purposes used synonymously with rechargeable lithium ion battery, unless indicated otherwise. For the purposes of the present disclosure, the term “electrochemical energy store” also encompasses, in particular, electrochemical capacitors such as supercapacitors. Electrochemical capacitors, which in the literature are also referred to as supercapacitors, are electrochemical energy stores which compared to batteries display a higher power density and compared to conventional capacitors a higher energy density.

Preference is given to secondary electrochemical energy stores. The energy store is, in particular, a lithium ion battery. It was able to be shown that the solid electrolyte interphase formed on a graphite anode was stable over at least 300 cycles. This allows economical operation of rechargeable batteries and use of the electrolyte.

In particular, the energy store can comprise a compound of the general formula (1) and carbon, in particular graphite, as electrode material and/or a propylene carbonate-containing electrolyte. Preference may be given, for example, to a lithium ion battery which contains a cathode, a graphite anode, a separator and an electrolyte comprising a tetraalkoxyethane of the general formula (1), in particular 1,1,2,2-tetramethoxyethane or 1,1,2,2-tetraethoxyethane, and propylene carbonate in a weight ratio of 1:1 or mixtures containing 1,1,2,2-tetramethoxyethane and/or 1,1,2,2-tetraethoxyethane together with propylene carbonate and dimethyl carbonate in a weight ratio of 1:1:1 or 1:2:2 and also preferably 1 M LiTFSI, LiFSI or LiPF₆.

In principle, it is possible to use all electrolytes, solvents, electrolyte salts and counterelectrodes which are known to those skilled in the art and are normally used in energy stores such as lithium ion batteries. For example, lithium metal, lithium titanate spinel (LTO) and carbon, in particular graphite, can be used as anode material and lithium iron phosphate (LFP) and lithium-nickel-manganese-cobalt oxide (NMC) can be used as cathode material.

The disclosure, according to an embodiment, further provides a method for forming a solid electrolyte interphase on an electrode of an electrochemical cell comprising an anode, a cathode and an electrolyte, wherein the cell is operated using the electrolyte of the disclosure.

The disclosure further provides for the use of a compound of the general formula (1) as indicated below:

[chemical structure]

where:
R¹, R², R³, R⁴ are identical or different and are selected independently from the group comprising linear or branched C₁₋₆-alkyl, C₁₋₆-alkenyl, C₁₋₆-alkynyl, C₃₋₆-cycloalkyl and phenyl, in each case unsubstituted or singly or multiply substituted by a substituent selected from the group comprising F, CN and C₁₋₂-alkyl singly or multiply substituted by fluorine, in an energy store, in particular an electrochemical energy store selected from the group comprising lithium battery, lithium ion battery, rechargeable lithium ion battery, lithium polymer battery, lithium ion capacitor and a supercapacitor.

The compound of the general formula (1) can be advantageously used, according to an embodiment, as electrolyte additive, solvent or cosolvent, especially in electrolytes which without addition of an additive do not form an effective SEI. In particular, the compound of the general formula (1) can be advantageously used, according to an embodiment, in an energy store which comprises carbon, in particular graphite, as electrode material and/or a propylene carbonate-containing electrolyte. For a description of the compound of the general formula (1), reference may be made to the above description. Particular preference may be given to 1,1,2,2-tetramethoxyethane and 1,1,2,2-tetraethoxyethane.

### BRIEF DESCRIPTION OF THE FIGURES

Examples and figures which serve to illustrate the present disclosure are presented below.

Here, the figures show:

FIG. 1 the reductive stability window of an electrolyte containing 1 M LiTFSI in a mixture of 1,1,2,2-tetramethoxyethane (TME) and propylene carbonate (PC) in FIG. 1a) and the reductive stability window of an electrolyte containing 1 M LiTFSI in a mixture of 1,1,2,2-tetraethoxyethane (TEE) and propylene carbonate in FIG. 1b). In each case, the current is plotted against the potential.

FIG. 2 the oxidative stability window in Pt/Li half cells of electrolytes each containing 1 M LiTFSI in mixtures of 1,1,2,2-tetraethoxyethane or 1,1,2,2-tetramethoxyethane and propylene carbonate and of 1 M LiFSI in a mixture of PC and 1,1,2,2-tetramethoxyethane. The current density is plotted against the potential.

FIG. 3 the oxidative stability window in an LiMn₂O₄/Li half cell for an electrolyte containing 1 M LiFSI in a mixture of propylene carbonate and 1,1,2,2-tetraethoxyethane.

FIG. 4 the charging and discharging capacity (left-hand ordinate axis) and Coulombic efficiency (right-hand ordinate axis) versus the number of charging/discharging cycles for an electrolyte containing 1 M LiTFSI in a 1:1 mixture of propylene carbonate and 1,1,2,2-tetraethoxyethane for a graphite/Li cell.

FIG. 5 the charging and discharging capacity and Coulombic efficiency versus the number of charging/discharging cycles for an electrolyte containing 1 M LiTFSI in a 1:1 mixture of propylene carbonate and 1,1,2,2-tetraethoxyethane in an LFP/graphite full cell.

FIG. 6 the charging and discharging capacity and Coulombic efficiency versus the number of charging/discharging cycles for an electrolyte containing 1 M LiFSI in a 1:1 mixture of propylene carbonate and 1,1,2,2-tetraethoxyethane in an NMC/graphite full cell.

FIG. 7 in FIG. 7a), the course of the cell voltage versus the capacity of the first cycle for an electrolyte containing 1 M LiTFSI in a 1:1 mixture of propylene carbonate and 1,1,2,2-tetraethoxyethane. FIG. 7b) shows a scanning electron micrograph of the cross section of secondary graphite particles of the surface after one cycle in this electrolyte.

FIG. 8 in FIG. 8a), the course of the cell voltage versus the time of the first cycle for an electrolyte containing 1 M LiPF₆ in propylene carbonate containing 2% by weight of FEC. FIG. 8b) shows a scanning electron micrograph of the graphite surface after one cycle in the electrolyte.

### DETAILED DESCRIPTION

### Example 1

### Determination of the Conductivity of 1,1,2,2-Tetraethoxyethane in Various Electrolytes

The conductivity of a 1 M solution of LiTFSI (lithium bis(trifluoromethanesulfonyl)imide (LiN(SO₂CF₃)₂) was determined in 1,1,2,2-tetraethoxyethane and in mixtures of 1,1,2,2-tetraethoxyethane (TEE), propylene carbonate (PC) and dimethyl carbonate (DMC).

To produce the electrolytes, 1,1,2,2-tetraethoxyethane, a mixture of 50% by weight of 1,1,2,2-tetraethoxyethane and 50% by weight of propylene carbonate or a mixture of 1,1,2,2-tetraethoxyethane, propylene carbonate and dimethyl carbonate in a weight ratio of 1:1:1 were initially charged. The respective required amount of LiTFSI or LiFSI (LiN(SO₂F)₂) was dissolved in these so that a concentration of 1 M of the lithium salt was obtained. In the same way, comparative electrolytes containing 1 M LiTFSI or LiPF₆ in propylene carbonate were produced.

The conductivity of the electrolytes was examined in a temperature range from −35° C. to +60° C. using a 2-electrode conductivity measurement cell (RHD Instruments, GC/Pt). For this purpose, the conductivity measurement cells were firstly heated to 60° C. and cooled in temperature steps of 10° C. to −30° C. and subsequently to −35° C. Table 1 below shows the conductivity in the temperature range from −35° C. to +60° C. in the corresponding solvent mixtures.

TABLE 1
Conductivity of 1M LiTFSI and LiFSI in various mixtures
containing 1,1,2,2-tetraethoxyethane (TEE)

| | | LiTFSI in | LiTFSI in | LiFSI in |
|---|---|---|---|---|
| | LiTFSI in | TEE:PC | TEE:PC:DMC | TEE:PC |
| T | TEE | (1:1 w/w) | (1:1:1 w/w) | (1:1 w/w) |
| [° C.] | [σ/mS cm⁻¹] | [σ/mS cm⁻¹] | [σ/mS cm⁻¹] | [σ/mS cm⁻¹] |
| −35 | 0.3 | 0.3 | 0.8 | 0.3 |
| −30 | 0.4 | 0.5 | 1.0 | 0.5 |
| −20 | 0.7 | 0.8 | 1.6 | 0.9 |
| −10 | 1.0 | 1.3 | 2.3 | 1.5 |
| 0 | 1.4 | 1.9 | 3.2 | 2.2 |
| 10 | 1.8 | 2.6 | 4.1 | 3.1 |
| 20 | 2.2 | 3.4 | 5.1 | 4.1 |
| 30 | 2.7 | 4.2 | 6.1 | 5.2 |
| 40 | 3.2 | 5.2 | 7.1 | 6.5 |
| 50 | 3.7 | 6.2 | 8.2 | 7.7 |
| 60 | 4.2 | 7.2 | 9.3 | 9.0 |

As can be seen from table 1, 1 M LiTFSI in 1,1,2,2-tetraethoxyethane (TEE) as sole solvent displays a conductivity at 20° C. of 2.2 mS cm⁻¹, which is below the comparative value of 4.1 mS cm⁻¹ in propylene carbonate. An addition of propylene carbonate led to a significant increase in the conductivity, while a mixture of TEE, PC and DMC displayed a conductivity which even slightly exceeded that of the comparative system 1 M LiPF₆ in PC of 5.0 mS cm⁻¹.

### Example 2

### Determination of the Conductivity of 1,1,2,2-Tetramethoxyethane in Various Electrolytes

The conductivity of electrolytes containing 1,1,2,2-tetramethoxyethane (TME) was examined in a temperature range from −35° C. to +60° C. as described in example 1 using a 2-electrode conductivity measurement cell (RHD Instruments, GC/Pt).

The conductivity of a 1 M solution of LiTFSI in 1,1,2,2-tetramethoxyethane (TME) and in mixtures of in each case 50% by weight of TME and PC and also mixtures of TME, PC and DMC in a weight ratio of 1:1:1 and 1:2:2 was determined. Table 2 below shows the conductivity in the temperature range from −35° C. to +60° C. in the corresponding solvents.

TABLE 2
Conductivity of 1M LiTFSI in various mixtures
containing 1,1,2,2-tetramethoxyethane (TME)

| | | LiTFSI in | LiTFSI in | LiTFSI in |
|---|---|---|---|---|
| | LiTFSI in | TME:PC | TME:PC:DMC | TME:PC:DMC |
| T | TME | (1:1 w/w) | (1:1:1 w/w) | (1:2:2 w/w) |
| [° C.] | [σ/mS cm⁻¹] | [σ/mS cm⁻¹] | [σ/mS cm⁻¹] | [σ/mS cm⁻¹] |
| −35 | 0.2 | 0.4 | 0.8 | 1.0 |
| −30 | 0.3 | 0.6 | 1.1 | 1.5 |
| −20 | 0.5 | 1.1 | 1.7 | 2.3 |
| −10 | 0.8 | 1.7 | 2.6 | 3.2 |
| 0 | 1.1 | 2.4 | 3.6 | 4.3 |
| 10 | 1.4 | 3.3 | 4.6 | 5.5 |
| 20 | 1.8 | 4.4 | 5.8 | 6.8 |
| 30 | 2.3 | 5.5 | 6.9 | 8.1 |
| 40 | 2.8 | 6.7 | 8.2 | 9.4 |
| 50 | 3.3 | 8.0 | 9.5 | 10.7 |
| 60 | 3.8 | 9.4 | 10.9 | 12.0 |

As can be seen from table 2, 1 M LiTFSI in 1,1,2,2-tetramethoxyethane (TME) as sole solvent displays a conductivity at 20° C. of 1.8 mS cm⁻¹, which is somewhat lower than the conductivity of 1,1,2,2-tetraethoxyethane. An addition of propylene carbonate and DMC led to a significant increase in the conductivity.

### Example 3

### Determination of the Reductive Electrochemical Stability and Cyclability of 1,1,2,2-Tetramethoxyethane and 1,1,2,2-Tetraethoxyethane Using a Graphite Electrode

The determination of the stability of the electrolytes in half cells was carried out by means of cyclic voltammetry. In this method, the electrode voltage is continuously changed cyclically. A three-electrode cell (Swagelok® type) having a graphite composite electrode (96%, 350 mAh/g; 1.1 mAh cm⁻²) as working electrode and lithium foil as counterelectrode and reference electrode was used for this purpose. A glass fiber nonwoven was used as separator.

To determine the reductive stability and cyclability, the potential between working electrode and reference electrode was firstly lowered from the equilibrium potential (OCP) to 0.025 V vs. Li/Li⁺ and subsequently increased again from 0.025 V to 1.5 V vs. Li/Li⁺. The cyclic potential change procedure between 0.025 V and 1.5 V vs. Li/Li⁺ was repeated twice. The rate of advance was 0.025 mV s⁻¹.

Two electrolytes each containing 1 M LiTFSI in mixtures of 1,1,2,2-tetraethoxyethane and propylene carbonate (1 M LiTFSI, PC:TEE (1:1)) or 1,1,2,2-tetramethoxyethane and propylene carbonate (1 M LiTFSI, PC:TME (1:1)) were examined. The electrolytes were produced by dissolving the required amount of LiTFSI in TEE or TME. FIG. 1a) shows the reductive stability window of the electrolyte containing 50% by weight of 1,1,2,2-tetramethoxyethane (TME) and FIG. 1b) shows the reductive stability window of the electrolyte containing 50% by weight of 1,1,2,2-tetraethoxyethane (TEE). The current is in each case plotted against the potential over three cycles. As can be seen from FIGS. 1a) and 1b), the electrolytes containing 50% by weight of propylene carbonate were stable and compatible with graphite electrodes. This shows that effective passivation of graphite can be achieved by means of 1,1,2,2-tetramethoxyethane and 1,1,2,2-tetraethoxyethane even in a 1:1 mixture with propylene carbonate. Reductive decomposition was not discernible for TME and TEE from the cyclic voltammogram.

### Example 4

### Determination of the Oxidative Electrochemical Stability of 1,1,2,2-Tetramethoxyethane and 1,1,2,2-Tetraethoxyethane Using a Platinum Electrode

The determination of the oxidative stability of the electrolytes in half cells was carried out by means of linear sweep voltammetry in a three-electrode cell of the Swagelok® type having a platinum electrode (Ø=0.1 cm, eDAQ) as working electrode and lithium foil as counterelectrode and reference electrode. A glass fiber nonwoven was used as separator. To determine the oxidative stability, the potential between working electrode and reference electrode was increased from the open-circuit voltage to 7.0 V vs. Li/Li⁺. The rate of advance was 0.1 mV s⁻¹.

Three electrolytes each containing 1 M LiTFSI in mixtures of 1,1,2,2-tetraethoxyethane and propylene carbonate (1 M LiTFSI, PC:TEE (1:1)) or 1,1,2,2-tetramethoxyethane and propylene carbonate (1 M LiTFSI, PC:TME (1:1)) and also 1 M LiFSI in a mixture of PC and TME (1:1) were examined. The electrolytes were produced by dissolving the required amount of LiTFSI or LiFSI in TEE or TME. FIG. 2 shows the oxidative stability window of the electrolytes. The current is plotted against the potential. As can be seen from FIG. 2, the electrolytes were stable up to a potential of 5 V vs. Li/Li⁺.

### Example 5

### Determination of the Oxidative Stability of 1,1,2,2-Tetraethoxyethane Using an LMO Electrode

The oxidative stability of an electrolyte containing 1 M LiFSI in a mixture of 1,1,2,2-tetraethoxyethane and propylene carbonate (1 M LiFSI, PC:TEE (1:1)) was examined using lithium-manganese oxide as working electrode. The determination of the oxidative stability was carried out as described in example 4 by means of linear sweep voltammetry in a three-electrode cell of the Swagelok® type. Lithium foil served as reference electrode and counterelectrode, and the potential between working electrode and reference electrode was increased from the open-circuit voltage to 4.9 V vs. Li/Li⁺. The rate of advance of the potential was 0.025 mV s⁻¹.

FIG. 3 shows the oxidative stability window of the electrolyte for a potential vs. Li/Li⁺ in the range from 3.2 V to 5 V. As can be seen from FIG. 3, complete delithiation without additional indications of parasitic Faradaic reactions was possible for the electrolyte based on a 1:1 mixture of 1,1,2,2-tetraethoxyethane and propylene carbonate through to a shut-off voltage of 4.3 V vs. Li/Li⁺.

### Example 6

### Examination of the Cycling Stability Using a Graphite Electrode

The examination of the cycling stability was carried out in a button cell construction (Hohsen Corp., CR2032) using lithium electrodes and graphite electrodes (MCMB). A glass fiber nonwoven was used as separator. Cycling was carried out in a voltage window from 0.025 V to 1.5 V. 3 formation cycles at 0.1 C and also 3 conditioning cycles at 0.25 C and 3 conditioning cycles at 0.5 C were carried out, followed by 41 charging/discharging cycles at 1.0 C. The measurements at constant current were carried out on a battery tester series 4000 (Maccor) at 20.0° C.±0.1° C.

An electrolyte containing 1 M LiTFSI in a mixture of 50% by weight of each of 1,1,2,2-tetraethoxyethane (TEE) and propylene carbonate (PC) was produced by initially charging the solvent mixture and dissolving the required amount of LiTFSI therein.

The charging and discharging capacity of the graphite/Li cell and also the Coulombic efficiency versus the number of cycles are shown in FIG. 4. As can be seen from FIG. 4, the electrolyte displayed a high Coulombic efficiency of 87.3% in the first cycle and a small capacity loss and a high Coulombic efficiency of >99.9% over the total period of cycling. This indicates effective passivation of the graphite surface by means of 1,1,2,2-tetraethoxyethane, even without addition of an SEI additive.

As comparative electrolytes, a solution of 1 M LiTFSI in propylene carbonate and also in propylene carbonate containing 5% by weight of the SEI additive vinylene carbonate were cycled in parallel. As expected, pure propylene carbonate displayed exfoliation of the graphite electrode after the first cycle. Reversible cycling was not possible. The addition of vinylene carbonate made cycling possible, but the cell displayed only a low Coulombic efficiency of 79.6% even in the first cycle and a rapid capacity loss within the first 20 cycles, which indicates that there is not effective passivation by vinylene carbonate. In contrast, the compound according to the disclosure displayed a high and constant Coulombic efficiency of >99.9% over the entire cycling time of 50 cycles examined.

### Example 7

### Examination of the Long-Term Cycling Stability in an LFP/Graphite Full Cell

The examination of the long-term cycling stability in full cells was likewise carried out in a button cell construction (Hohsen Corp., CR2032) using lithium iron phosphate (LFP, 83%, 150 mAh/g; 1.0 mAh/cm⁻²) and graphite electrodes (96%, 350 mAh/g; 1.1 mAh cm⁻²). A polymer nonwoven was used as separator. Cycling was carried out in a voltage window from 2.5 V to 3.6 V. 3 formation cycles at 0.1 C and 3 conditioning cycles at 0.33 C were carried out, followed by 320 charging/discharging cycles at 1.0 C. The measurements were carried out on a battery tester series 4000 (Maccor) at 20.0° C.±0.1° C.

An electrolyte containing 1 M LiTFSI in a mixture of 50% by weight each of 1,1,2,2-tetraethoxyethane (TEE) and propylene carbonate (PC) was used, with the solvent mixture being initially charged and the required amount of LiTFSI being dissolved therein.

FIG. 5 shows the discharging and charging capacity and also the Coulombic efficiency of the full cell versus the number of cycles. As can be seen from FIG. 5, the electrolyte displayed a Coulombic efficiency of 88.4% in the first cycle and a high Coulombic efficiency of >99.9% over 300 cycles. Furthermore, this result demonstrates that there is compatibility with LFP cathode material.

### Example 8

### Examination of the Cycling Stability in an NMC/Graphite Full Cell

The cycling stability in full cells was repeated as described in example 7 using a lithium-nickel_{0.5}-manganese_{0.3}-cobalt_{0.2} oxide cathode (NMC532) against graphite over 40 charging/discharging cycles at 1.0 C. Cycling was carried out in a voltage window from 2.8 V to 4.2 V. 1 M LiFSI in a 1:1 mixture of 1,1,2,2-tetraethoxyethane and propylene carbonate was used as electrolyte.

FIG. 6 shows the discharging and charging capacity and also the Coulombic efficiency of the NMC/graphite full cell versus the number of cycles. As can be seen from FIG. 6, the electrolyte displayed a Coulombic efficiency of 84.5% in the first cycle and a Coulombic efficiency of >99.5% over 40 cycles. This shows that there is also good compatibility with NMC cathode material. The electrolyte of the disclosure can thus also be used with cathode materials at a shut-off voltage of up to 4.2 V.

### Example 9

Examination of the Graphite Surface after Cycling in 1,1,2,2-Tetraethoxyethane Mixtures

To examine the passivation of the graphite electrode by 1,1,2,2-tetraethoxyethane, the surface of the electrode was examined by scanning electron microscopy after one charging/discharging cycle.

A graphite anode (96%, 350 mAh/g; 1.1 mAh cm⁻²) was cycled against a lithium iron phosphate cathode (LFP) or a lithium-nickel_{0.5}-manganese_{0.3}-cobalt_{0.2} oxide cathode (NMC532) in a full cell having a button cell construction (Hohsen Corp., CR2032). A polymer nonwoven was used as separator. The charging/discharging cycle was carried out in a voltage window from 2.5 V to 3.6 V (LFP) or from 2.8 V to 4.2 V (NMC532). The measurements were carried out at 250° C.±0.1° C. on a battery tester series 4000 (Maccor).

1 M LiTFSI in a 1:1 mixture of 1,1,2,2-tetraethoxyethane and propylene carbonate was used as electrolyte. A solution of 1 M LiPF₆ in propylene carbonate containing 2% by weight of the SEI additive fluoroethylene carbonate (FEC) was used as comparative electrolyte.

After the charging/discharging cycle had been carried out, the graphite electrodes were in each case removed from the cell and the surfaces were examined by high-resolution scanning electron microscopy (SEM) using a ZEISS Auriga® electron microscope.

FIG. 7a) shows the course of the cell voltage (graphite/LFP cell) versus the capacity of the first cycle for the electrolyte containing 50% by weight of 1,1,2,2-tetraethoxyethane and PC, and FIG. 7b) shows a scanning electron micrograph of the graphite surface (cross section of the secondary graphite particles). FIG. 7a) shows that a reversible intercalation/deintercalation of the Li⁺ ions in the graphite was possible in the first cycle. As can be seen from FIG. 7b), the surface of the graphite electrode was intact after the charging/discharging cycle had been carried out. There were no discernible signs of exfoliation.

FIG. 8a) shows the cell voltage of the comparative cell (graphite/NMC532, containing 1 M LiPF₆ in propylene carbonate containing 2% by weight of fluoroethylene carbonate as electrolyte) for the first cycle versus time. FIG. 8b) shows a scanning electron micrograph of the graphite surface after the charging/discharging cycle. As can be seen from FIG. 8a), a significantly lower reversibility of the intercalation/deintercalation of Li⁺ ions in the graphite is observed. FIG. 8b) clearly shows that the surface of the graphite electrode displayed severe exfoliation after one charging/discharging cycle in propylene carbonate even when using the SEI additive FEC.

Comparison of FIGS. 7b) and 8b) confirms effective passivation by 1,1,2,2-tetraethoxyethane which displayed significantly better protection of the graphite electrode than the use of a conventional SEI additive.

Overall, the results show that 1,1,2,2-tetraethoxyethane and 1,1,2,2-tetramethoxyethane can form a passivating protective layer which conducts lithium ions on the surface of graphite. In addition, the two compounds display satisfactory conductivity and good oxidative stability. Furthermore, the compounds were able to be operated stably in lithium ion batteries with good cycling stability over 300 cycles.

The disclosure forming the basis of the present patent application arose in a project supported by BMBF under the support number 3120034900.

It is to be understood that the foregoing is a description of one or more preferred exemplary embodiments of the invention. The invention is not limited to the particular embodiment(s) disclosed herein, but rather is defined solely by the claims. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

1. An electrolyte for an energy store, comprising an electrolyte salt and a solvent, wherein the solvent comprises at least one compound of the general formula (1) as indicated below: [chemical structure]
where:
R¹, R², R³, R⁴ are identical or different and selected independently from the group comprising linear or branched C₁₋₆-alkyl, C₂₋₆-alkenyl, C₂₋₆-alkynyl, C₃₋₆-cycloalkyl and phenyl, in each case unsubstituted or singly or multiply substituted by a substituent selected from the group comprising F, CN and C₁₋₂-alkyl singly or multiply substituted with fluorine.

2. The electrolyte as claimed in claim 1, wherein R¹, R², R³, R⁴ are identical or different and selected independently from the group comprising unsubstituted C₁-C₅-alkyl or phenyl and C₁-C₅-alkyl or phenyl singly or multiply substituted by fluorine, CN or CF₃.

3. The electrolyte as claimed in claim 1, wherein R¹, R², R³, R⁴ are identical or different and selected independently from the group comprising methyl, ethyl, n-propyl and isopropyl.

4. The electrolyte as claimed in claim 1, wherein the compound of the general formula (1) is selected from the group comprising 1,1,2,2-tetramethoxyethane and 1,1,2,2-tetraethoxyethane.

5. The electrolyte as claimed in claim 1, wherein the solvent comprises the compound of the formula (1) in an amount of from ≥0.1% by weight to ≤100% by weight, preferably in an amount of from ≥10% by weight to ≤80% by weight, more preferably in an amount of from ≥20% by weight to ≤50% by weight, particularly preferably in an amount of from ≥30% by weight to ≤50% by weight, based on the total weight of the electrolyte solvent.

6. The electrolyte as claimed in claim 1, wherein the electrolyte comprises an organic solvent selected from the group comprising ethylene carbonate, ethyl methyl carbonate, propylene carbonate, dimethyl carbonate, diethyl carbonate, acetonitrile, propionitrile, 3-methoxypropionitrile, glutaronitrile, adiponitrile, pimelonitrile, gamma-butyrolactone, gamma-valerolactone, dimethoxyethane, 1,3-dioxolan, methyl acetate, ethyl acetate, ethyl methanesulfonate, dimethyl methylphosphonate, linear or cyclic sulfone, symmetrical or unsymmetrical alkyl phosphates and mixtures thereof.

7. The electrolyte as claimed in claim 6, wherein the solvent is selected from the group comprising propylene carbonate, ethylene carbonate, ethyl methyl carbonate, dimethyl carbonate, diethyl carbonate and mixtures thereof.

8. An energy store, in particular electrochemical energy store selected from the group comprising lithium battery, lithium ion battery, rechargeable lithium ion battery, lithium polymer battery, lithium ion capacitor or supercapacitor, comprising an electrolyte as claimed in claim 1.

9. A method for forming a solid electrolyte interphase on an electrode of an electrochemical cell comprising an anode, a cathode and an electrolyte, wherein the cell is operated using an electrolyte as claimed in claim 1.

10. The use of a compound of the general formula (1) as indicated below: [chemical structure]
where:
R¹, R², R³, R⁴ are identical or different and are selected independently from the group comprising linear or branched C₁₋₆-alkyl, C₁₋₆-alkenyl, C₁₋₆-alkynyl, C₃₋₆-cycloalkyl and phenyl, in each case unsubstituted or singly or multiply substituted by a substituent selected from the group comprising F, CN and C₁₋₂-alkyl singly or multiply substituted by fluorine,
in an energy store, in particular an electrochemical energy store selected from the group comprising lithium battery, lithium ion battery, rechargeable lithium ion battery, lithium polymer battery, lithium ion capacitor and a supercapacitor.
